(19) United States
(12) Patent Application Publication
(10) Pub. No.: US 2024/0252024 A1
(43) Pub. Date: Aug. 1, 2024

(54) DISPOSABLE OUTER SHEATH MECHANISM FOR ENDOSCOPE

(71) Applicant: Chang-Bih SHIE, Tainan City (TW)

(72) Inventor: Chang-Bih SHIE, Tainan City (TW)

(21) Appl. No.: 18/565,451

(22) PCT Filed: Jun. 10, 2022
(86) PCT No.: PCT/CN2022/098036

§ 371 Date: Nov. 29, 2023

(30) Foreign Application Priority Data
Jun. 15, 2021 (CN) 202110659497.8

(51) Int. Cl. A61B 1/00

(52) U.S. Cl. CPC A61B 1/00142; A61B 1/00103; A61B 1/00128; A61B 1/00135

(57) ABSTRACT

A disposable outer sheath mechanism for an endoscope includes an outer sheath unit and a fixing unit. The endoscope includes a connecting head, and an insertion tube extending outwardly from the connecting head for insertion into a body of a patient. The outer sheath unit includes an outer sheath having an elongated tubular shape and configured to be detachably sleeved on the insertion tube. The fixing unit is fixed to the outer sheath and is configured to be detachably connected to the connecting head.

### FIELD

The disclosure relates to a protective member for an endoscope, more particularly to a disposable outer sheath mechanism for an endoscope.

### BACKGROUND

Endoscope is a common invasive surgical instrument at present, which can invade the body of a patient with an insertion tube to perform symptom inspection and microsurgery. Since the insertion tube will directly contact body fluids of the patient, after the endoscope is used, it must be disinfected and sterilized to eliminate possible sources of infection, such as bacteria and viruses, so that it can be reused to reduce medical costs. However, when the endoscope is used for patients with certain high risk infectious diseases, such as AIDS patients or patients with new coronary pneumonia, there will be doubts whether the endoscope is completely sterilized in the general disinfection operation after the endoscope is used, so that the disinfection operation with higher safety standards must be adopted, thereby resulting in high maintenance cost of medical equipment. If the endoscope is discarded after use, it will increase medical costs.

### SUMMARY

Therefore, an object of the present disclosure is to provide a disposable outer sheath mechanism for an endoscope that can alleviate at least one of the drawbacks of the prior art.

According to one aspect of this disclosure, a disposable outer sheath mechanism for an endoscope includes a fixing unit, an outer sheath unit, and a connecting unit. The endoscope includes a connecting head, and an insertion tube extending outwardly from the connecting head for insertion into a body of a patient. The fixing unit includes a socket tube configured to be sleeved on and detachably connected to the connecting head, and a fixing module sleeved on the socket tube for fixing the socket tube to the connecting head. The outer sheath unit includes an outer sheath having an elongated tubular shape and configured to be detachably sleeved on the insertion tube. The connecting unit includes a first connecting member disposed on one of the socket tube and the outer sheath, and a second connecting member disposed on the other one of the socket tube and the outer sheath for detachable connection with the first connecting member.

According to another aspect of this disclosure, a disposable outer sheath mechanism for an endoscope includes an outer sheath unit and a fixing unit. The endoscope includes a connecting head, and an insertion tube extending outwardly from the connecting head for insertion into a body of a patient. The outer sheath unit includes an outer sheath having an elongated tubular shape and configured to be detachably sleeved on the insertion tube. The fixing unit is fixed to the outer sheath and is configured to be detachably connected to the connecting head.

### BRIEF DESCRIPTION OF THE DRAWINGS

Other features and advantages of the disclosure will become apparent in the following detailed description of the embodiments with reference to the accompanying drawings, of which:

FIG. 1 is a fragmentary perspective view of an endoscope incorporating a disposable outer sheath mechanism according to the first embodiment of the present disclosure;

FIG. 2 is a fragmentary exploded perspective view of the endoscope and the first embodiment;

FIG. 3 is a fragmentary partly exploded sectional view, illustrating how the first embodiment is installed on the endoscope;

FIG. 4 is a fragmentary assembled sectional view, illustrating the first embodiment after being installed on the endoscope;

FIG. 5 is a view similar to FIG. 2, but illustrating an alternative form of a fixing module of the first embodiment;

FIG. 6 is an assembled perspective view of FIG. 5;

FIG. 7 is a fragmentary perspective view of a disposable outer sheath mechanism according to the second embodiment of the present disclosure installed on the endoscope;

FIG. 8 is an exploded perspective view of FIG. 7;

FIG. 9 is a fragmentary assembled sectional view, illustrating the second embodiment after being installed on the endoscope;

FIG. 10 is a view similar to FIG. 8, but illustrating an alternative form of a connecting unit of the second embodiment;

FIG. 11 is a fragmentary exploded perspective view of the endoscope and a disposable outer sheath mechanism according to the third embodiment of the present disclosure;

FIG. 12 is an exploded partly sectional view of FIG. 11;

FIG. 13 illustrates an assembly of FIG. 12;

FIG. 14 illustrates an alternative form of a fixing unit of the third embodiment;

FIG. 15 is a fragmentary exploded perspective view of the endoscope and a disposable outer sheath mechanism according to the fourth embodiment of the present disclosure;

FIG. 16 is an assembled sectional view of FIG. 15;

FIG. 17 is a fragmentary exploded perspective view of the endoscope and a disposable outer sheath mechanism according to the fifth embodiment of the present disclosure;

FIG. 18 is an assembled sectional view of FIG. 17;

FIG. 19 is a fragmentary perspective view of an outer sheath unit of a disposable outer sheath mechanism according to the sixth embodiment of the present disclosure which can match with two different types of end ring modules;

FIG. 20 is a fragmentary sectional view, illustrating the sixth embodiment using one type of the end ring modules; and

FIG. 21 is a fragmentary sectional view, illustrating the sixth embodiment using the other type of the end ring modules.

### DETAILED DESCRIPTION

Before the present disclosure is described in greater detail with reference to the accompanying drawings and embodiments, it should be noted herein that like elements are denoted by the same reference numerals throughout the disclosure.

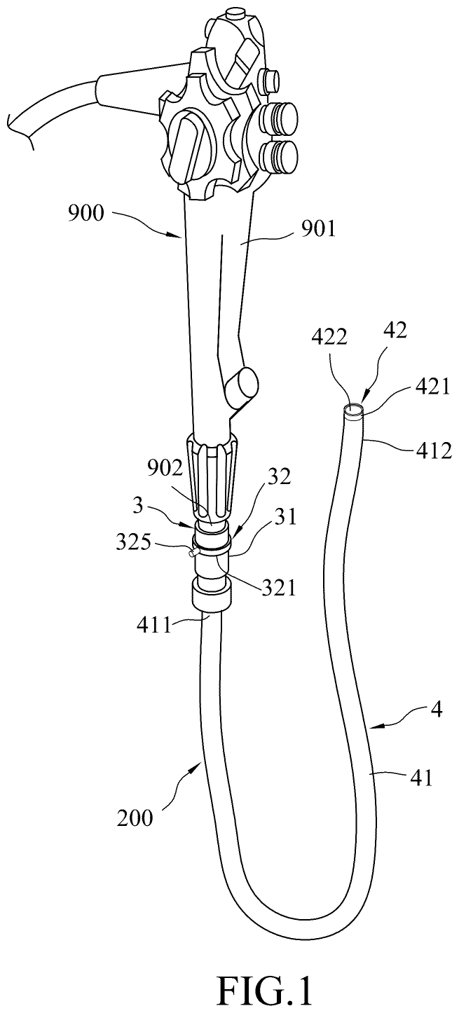

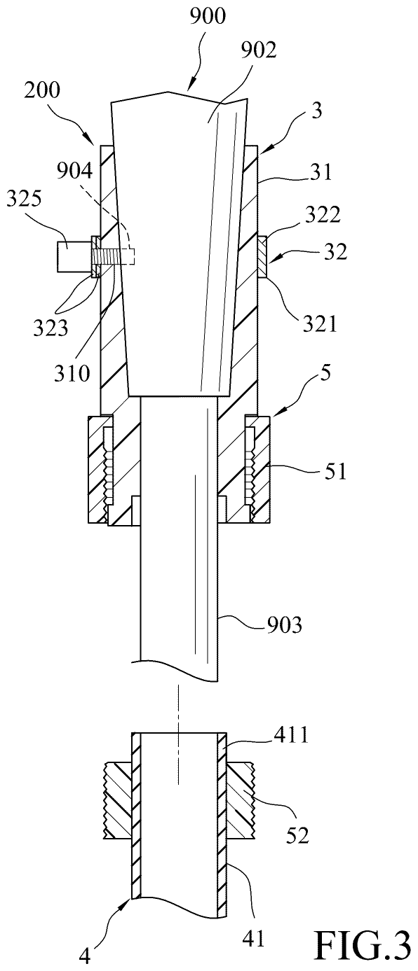

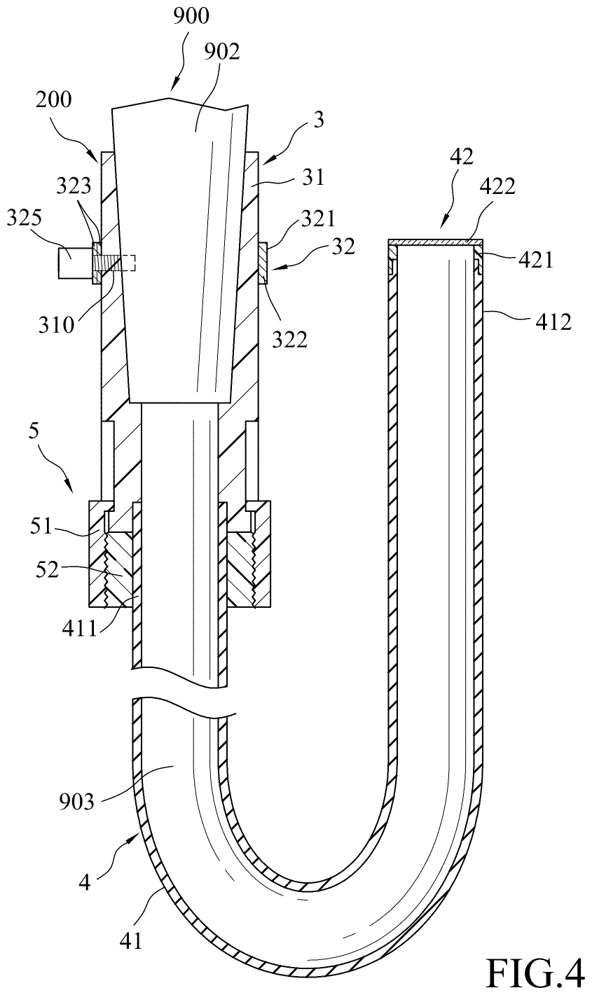

Referring to FIGS. 1 to 4, a disposable outer sheath mechanism 200 according to the first embodiment of the present disclosure is suitable to be detachably installed on an endoscope 900, and includes a fixing unit 3, an outer sheath unit 4, and a connecting unit 5. The endoscope 900 generally includes a handle 901 for holding and operating, a connecting head 902 connected to the handle 901, and an insertion tube 903 extending outwardly from the connecting head 902 for insertion into a body of a patient. An outer surface of the connecting head 902 is formed with a threaded hole 904.

The fixing unit 3 includes a socket tube 31 configured to be sleeved on and detachably connected to the connecting head 902, and a fixing module 32 sleeved on the socket tube 31 for fixing the socket tube 31 to the connecting head 902. The socket tube 31 has a threaded hole 310 extending radially from an inner surface to an outer surface thereof for communicating with the threaded hole 904 of the connecting head 902.

The fixing module 32 includes a socket ring 321 sleeved on the socket tube 31, and a fixing member 325 detachably and threadedly connected to the socket ring 321. The socket ring 321 has a C-shaped ring body 322 sleeved on the socket tube 31, and two mating portions 323 respectively protruding from two ends of the ring body 322 toward each other. Each mating portion 323 is formed with a threaded hole 324. When the mating portions 323 are brought to overlap each other, a diameter of the ring body 322 is reduced, so that the ring body 322 can elastically compress the socket tube 31. After the mating portions 323 are overlapped with each other, the threaded holes 324 of the mating portions 323 are aligned with the threaded holes 310 and 904 of the socket tube 31 and the connecting head 902, and the fixing member 325 is threaded through the threaded holes 324 of the mating portions 323 and the threaded hole 310 of the socket tube 31, and then threadedly engages the threaded hole 904 of the connecting head 902, thereby fixing the socket ring 321 to the socket tube 31 and simultaneously tightly fixing the socket tube 31 to the connecting head 902.

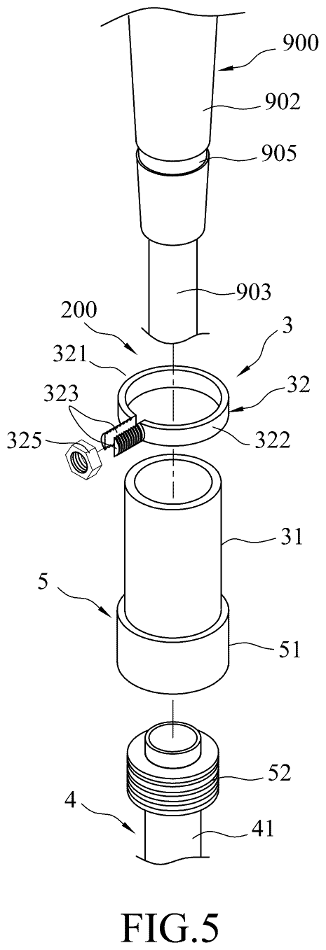

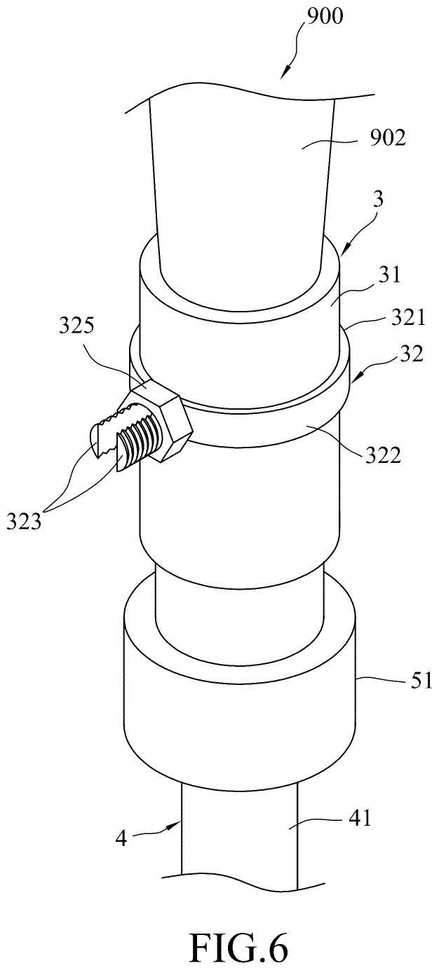

There are many types of fixing modules 32 that can be used to fix the socket tube 31 to the connecting head 902, an example of which is shown in FIGS. 5 and 6. As shown in FIGS. 5 and 6, the mating portions 323 of the socket ring 321 are configured as protruding rods extending outwardly, radially and respectively from two ends of the ring body 322. The protruding rods or mating portions 323 have inner surfaces facing each other, and outer surfaces having threads. Further, the fixing member 325 is configured as a nut sleeved on the protruding rods 323 and threadedly connected to the threads thereof to tighten the ring body 322 around the socket tube 31. As such, the socket tube 31 is forced to be partially elastically deformed and positioned in an embedding groove 905 of the connecting head 902. Therefore, during implementation, the type of the fixing module 32 is not limited to what is disclosed herein.

With reference to FIGS. 1, 2 and 4, the outer sheath unit 4 includes an outer sheath 41 having an elongated tubular shape and configured to be detachably sleeved on the insertion tube 903, and an end ring module 42 fixed to the outer sheath 41 and configured to be positioned outside a distal end of the insertion tube 903 that is distal to the connecting head 902. The outer sheath 41 is made of a flexible and elastically deformable medical material, such as, but not limited to, medical grade silicone, and has a first end portion 411 configured to be adjacent to the connecting head 902, and a second end portion 412 configured to be adjacent to the distal end of the insertion tube 903.

The end ring module 42 includes a positioning end ring 421 sleeved fixedly on the second end portion 412 of the outer sheath 41, and a transparent cover 422 fixed to one end of the positioning end ring 421 for covering the distal end of the insertion tube 903. The transparent cover 422 enables the endoscope 900 to capture images, and is made of a material, such as, but not limited to, glass and plastic.

The connecting unit 5 interconnects the fixing unit 3 and the outer sheath unit 4. In this embodiment, the connecting unit 5 includes a first connecting member 51 sleeved axially and movably on the socket tube 31, and a second connecting member 52 sleeved fixedly on the first end portion 411 of the outer sheath 41. An inner peripheral surface of the first connecting member 51 is provided with threads. An outer peripheral surface of the second connecting member 52 is provided with threads. The first connecting member 51 is threadedly connected to the second connecting member 52, so that the first end portion 411 of the outer sheath 41 is inserted fixedly into the socket tube 31.

To assemble the disposable outer sheath mechanism 200 with the endoscope 900, the fixing unit 3 is first sleeved fixedly on the connecting head 902 of the endoscope 900, after which the outer sheath unit 4 is sleeved on the insertion tube 903, and the first connecting member 51 is then threadedly connected to the second connecting member 52, thereby completing the assembly of the disposable outer sheath mechanism 200 with the endoscope 900.

With the disposable outer sheath mechanism 200 assembled on the endoscope 900, and with the outer sheath unit 4 covering the insertion tube 903, the insertion tube 903 can be prevented from contacting body fluids of the patient. After using the endoscope 900, it is only necessary to detach the first connecting member 51 from the second connecting member 52, and the outer sheath unit 4 can be detached from the insertion tube 903 and be discarded as medical waste. Since the insertion tube 903 of the endoscope 900 is not in contact with the body fluids of the patient, the endoscope 900 can be reused after being treated by conventional disinfection and sterilization methods.

In this first embodiment, the end ring module 42 is provided with the transparent cover 422 which cooperates with the outer sheath 41 to close the insertion tube 903. However, during implementation, in other embodiments of this disclosure, the end ring module 42 may not be provided with the transparent cover 422, so that after the outer sheath unit 4 is sleeved on the insertion tube 903, the distal end of the insertion tube 903 may be exposed. Through this, a medical personnel can perform a surgical treatment on the patient through a suction line, a pump line, and a flushing line exposed at the distal end of the insertion tube 903 and a surgical instrument.

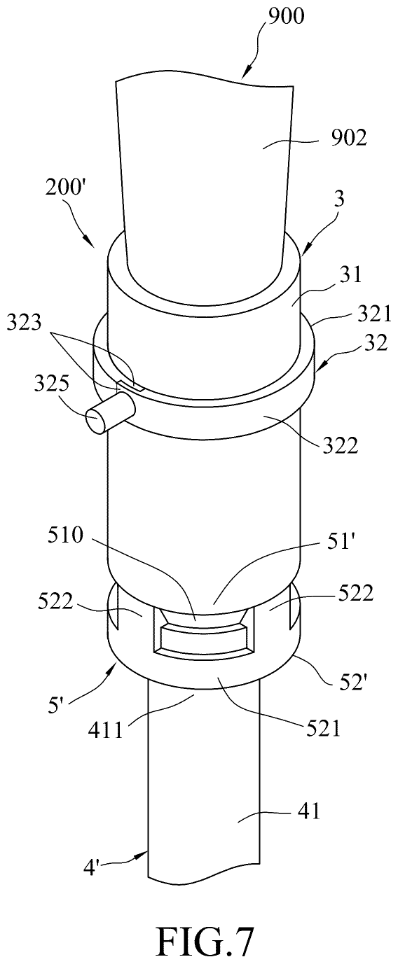

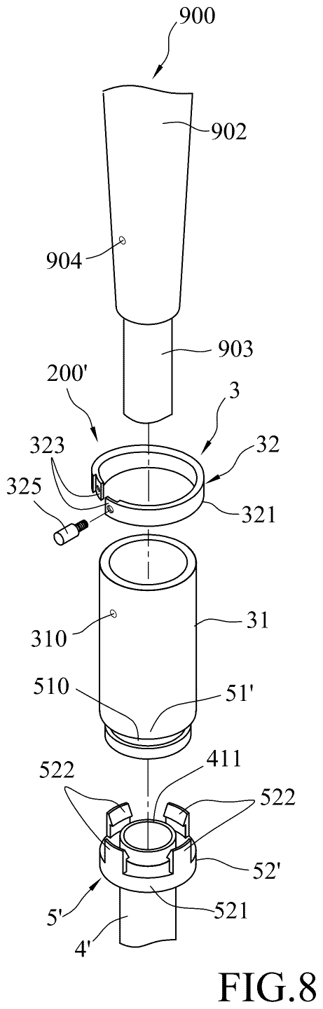

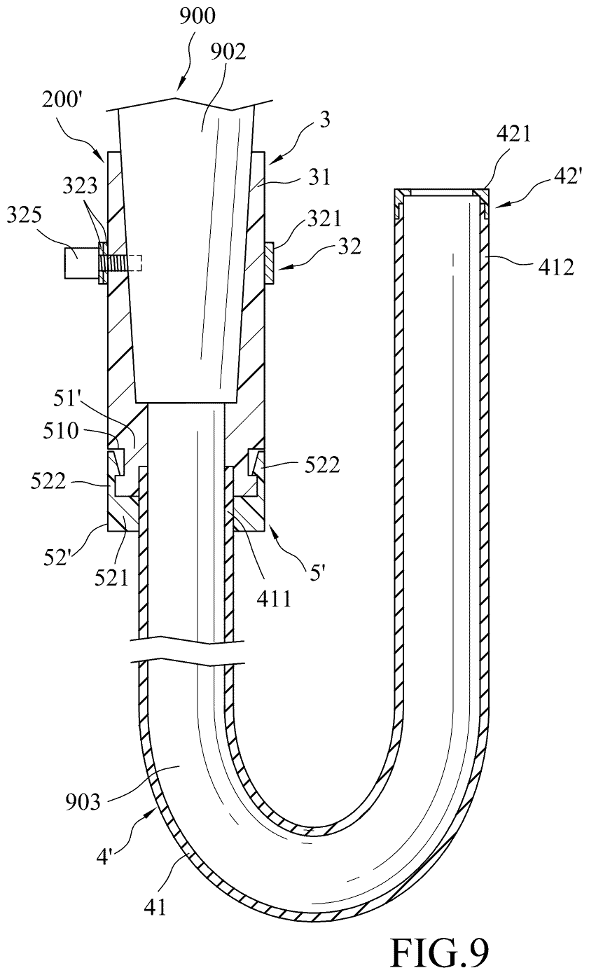

Referring to FIGS. 7 to 9, the second embodiment of the disposable outer sheath mechanism 200′ of this disclosure is identical to the first embodiment, but differs in the structural design of the outer sheath unit 4 and the connecting unit 5. In this embodiment, the end ring module 42′ of the outer sheath unit 4′ is not provided with the transparent cover 422 (see FIG. 1), and the positioning end ring 421 thereof is abuttingly positioned on a peripheral area of the distal end of the insertion tube 903, so that the distal end of the insertion tube 903 is exposed.

Further, in this embodiment, the first connecting member 51′ of the connecting unit 5′ is integrally provided at one end of the socket tube 31, and has an outer peripheral surface provided with an annular engaging groove 510. The second connecting member 52′ of the connecting unit 5′ has a connecting ring portion 521 sleeved fixedly on the first end portion 411 of the outer sheath 41, and a plurality of angularly spaced-apart hook portions 522 extending upwardly from the connecting ring portion 521 and facing the first end portion 411 for detachable engagement with the annular engaging groove 510.

When the fixing unit 3 is sleeved fixedly on the connecting head 902 of the endoscope 900 and the outer sheath unit 4′ is sleeved on the insertion tube 903, the hook portions 522 of the second connecting member 52′ are hooked to the annular engaging groove 510 of the first connecting member 51′ and are positioned thereat. Through this, the first end portion 411 of the outer sheath 41 is inserted and positioned in the socket tube 31.

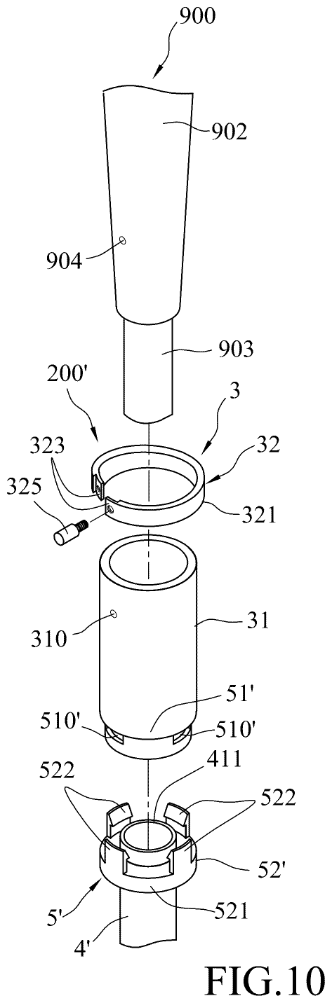

In this embodiment, the first connecting member 51′ is provided with only one annular engaging groove 510 for hooking engagement by the hook portions 522. However, during implementation, in other embodiments of this disclosure, the first connecting member 51′ may be provided with a plurality of angularly spaced-apart engaging grooves 510′, so that the hook portions 522 may respectively engage with the engaging grooves 510′, as shown in FIG. 10.

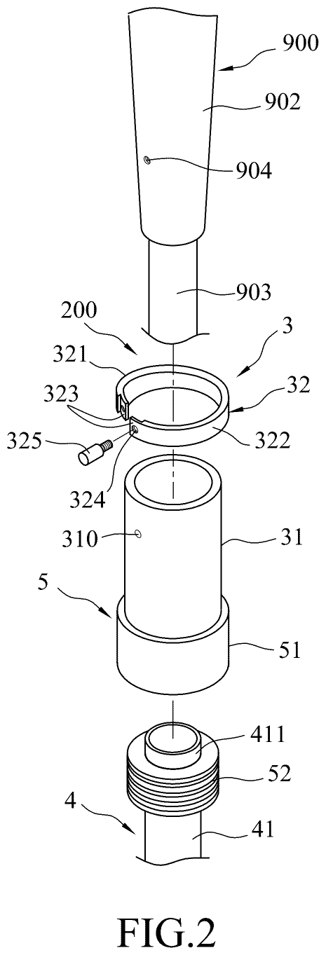

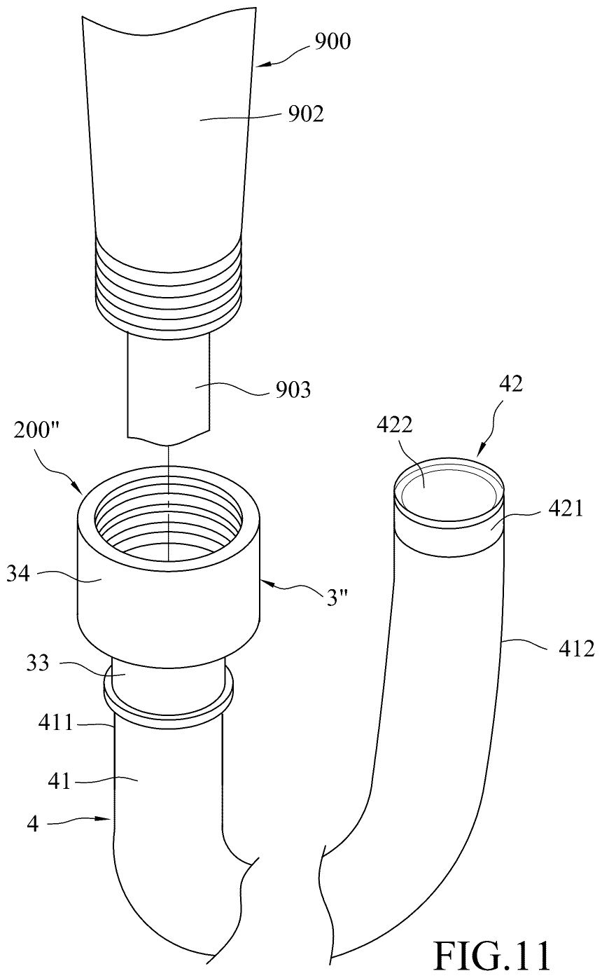

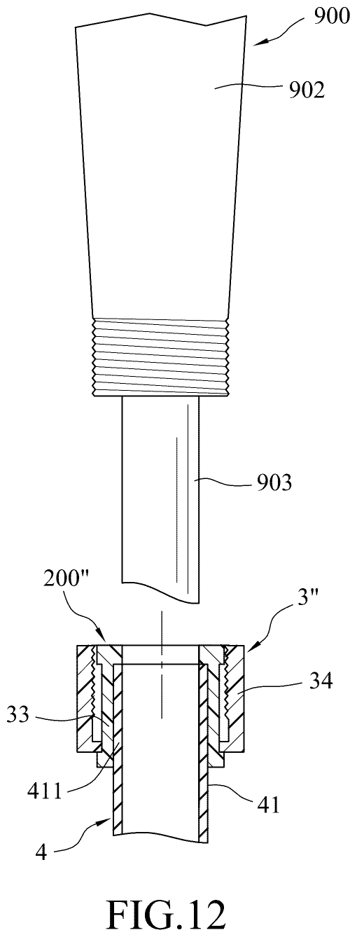

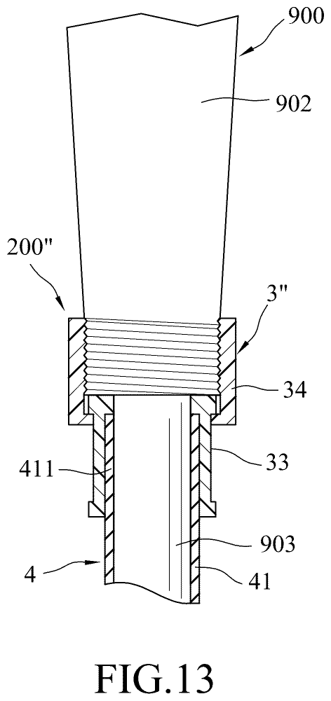

Referring to FIGS. 11 to 13, the third embodiment of the disposable outer sheath mechanism 200″ of this disclosure is identical to the first embodiment, but differs in the structural design of the fixing unit 3 and in the connection design of the fixing unit 3 and the outer sheath unit 4. Further, in this embodiment, the connecting unit 5 (see FIG. 2) is not provided.

The connecting head 902 of the endoscope 900 used in this embodiment has an outer peripheral surface provided with threads. The fixing unit 3″ of this embodiment includes a fixing ring 33 sleeved fixedly on the first end portion 411 of the outer sheath 41, and a threaded ring 34 sleeved on and axially movable relative to the fixing ring 33.

To assemble the disposable outer sheath mechanism 200″ with the endoscope 900, the outer sheath 41 is first sleeved on the insertion tube 903 of the endoscope 900, after which the threaded ring 34 is moved axially to protrude out of the fixing ring 33 and threadedly engage the connecting head 902, so that one end of the fixing ring 33 is abuttingly positioned on an end of the connecting head 902, thereby completing the assembly.

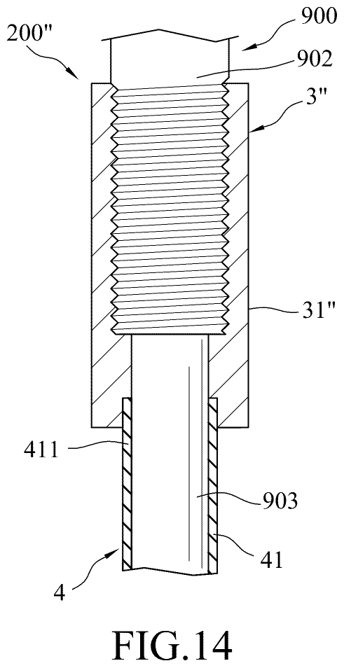

FIG. 14 illustrates an alternative form of the fixing unit 3″ of the third embodiment. As shown, the fixing unit 3″ includes a socket tube 31″ fixed to the first end portion 411 of the outer sheath 41 and having internal threads configured to be threadedly and detachably connected to the connecting head 902.

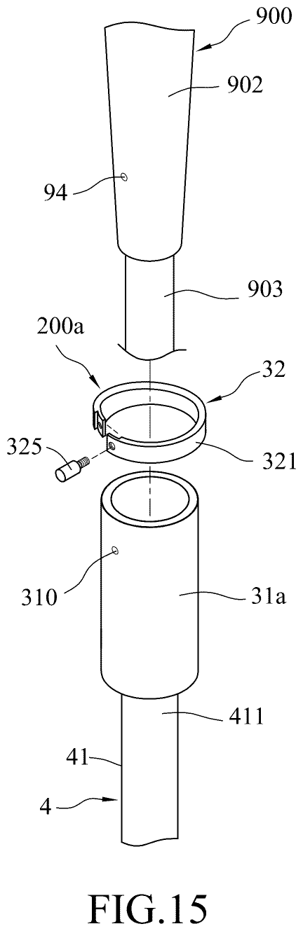

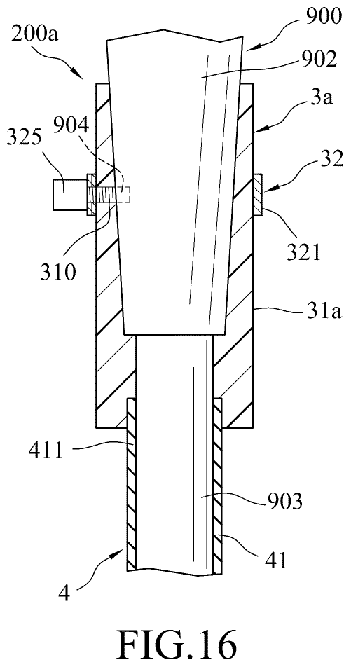

Referring to FIGS. 15 and 16, the fourth embodiment of the disposable outer sheath mechanism (200a) of this disclosure is identical to the first embodiment, but differs in the structural design of the fixing unit 3 and the outer sheath unit 4. Further, the connecting unit 5 (see FIG. 2) is not provided in this embodiment.

The socket tube (31a) of the fixing unit (3a) is fixed to the first end portion 411 of the outer sheath 41.

To assemble the disposable outer sheath mechanism (200a) with the endoscope 900, the insertion tube 903 is inserted through the socket tube (31a) and the outer sheath 41 until the socket tube (31a) is sleeved on the connecting head 902 and the first end portion 411 of the outer sheath 41 is sleeved on the insertion tube 903, after which the fixing module 32 is used to fix the socket tube 31a to the connecting head 902, thereby completing the assembly.

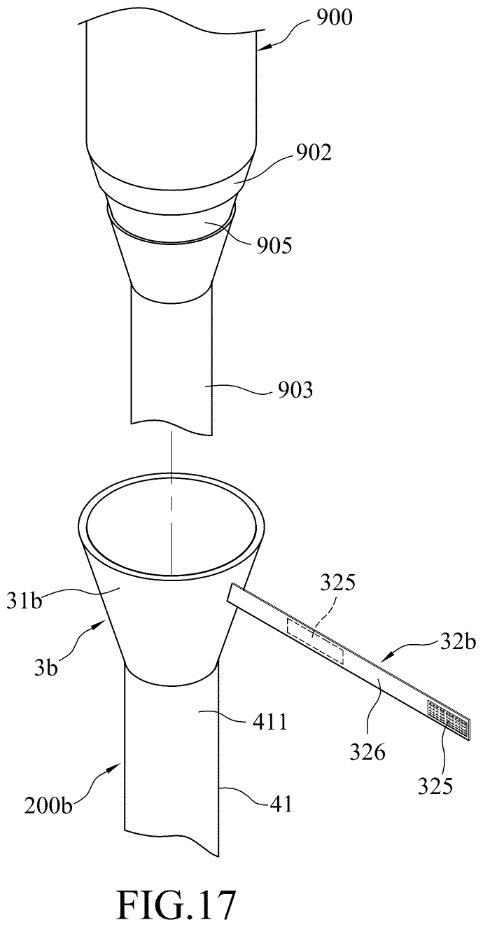

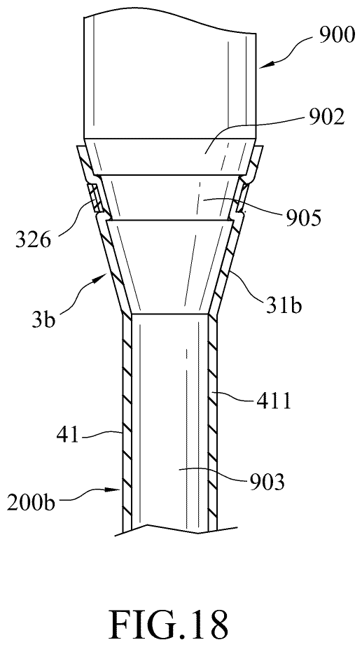

Referring to FIGS. 17 and 18, the fifth embodiment of the disposable outer sheath mechanism (200b) of this disclosure is identical to the fourth embodiment, but differs in the structural design of the fixing unit 3.

The connecting head 902 of the endoscope 900 used in this embodiment has an outer peripheral surface provided with an annular embedding groove 905. The socket tube (31b) of the fixing unit (3b) of this embodiment is connected end-to-end with the first end portion 411 of the outer sheath 41, and the fixing module (32b) thereof includes a fixing strap 326 having one end fixed to the socket tube (31b), and two fixing members (325b) respectively disposed on two opposite sides of the fixing strap 326. In this embodiment, the fixing members (325b) are hook and loop fasteners. However, during implementation, the fixing members (325b) may be changed to other forms of interlockable members, and are not limited to what is disclosed herein.

To assemble the disposable outer sheath mechanism (200b) with the endoscope 900, the outer sheath 41 is similarly sleeved on the insertion tube 903, and the socket tube (31b) is sleeved on the connecting head 902 of the endoscope 900, after which the fixing strap 326 is wrapped around the socket tube (31b) to force the socket tube (31b) to partially deform so as to be embedded in the embedding groove 902, and the fixing members 325 are then connected to each other for tightly fixing the socket tube (31b) to the connecting head 902, thereby completing the assembly.

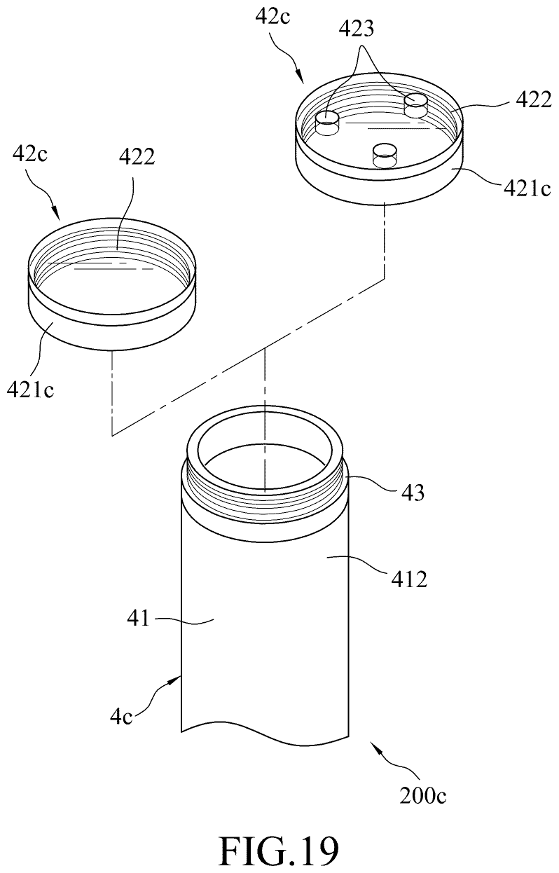

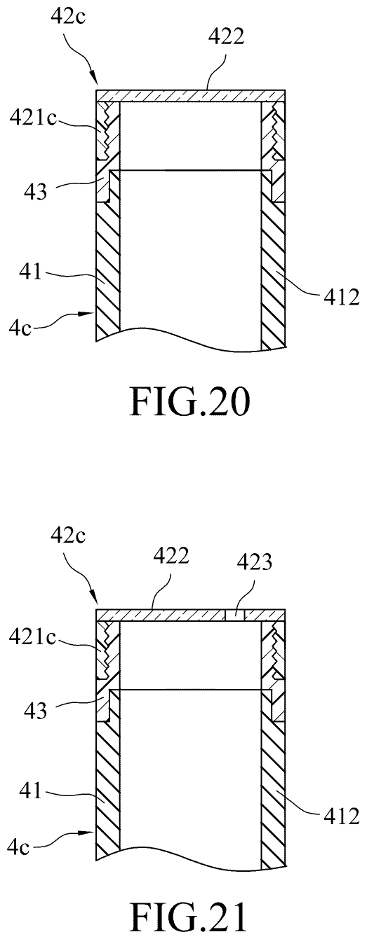

Referring to FIGS. 19 to 21, the sixth embodiment of the disposable outer sheath mechanism (200c) of this disclosure is identical to the first embodiment, but differs in the structural design of the outer sheath unit 4.

The outer sheath unit (4c) of this embodiment further includes an adapter ring 43 sleeved fixedly on the second end portion 412 of the outer sheath 41 and having an outer peripheral surface provided with threads. Two different types of the end ring modules (42c) of the outer sheath unit (4c) are also provided in this embodiment. The first type of the end ring modules (42c) includes a positioning end ring (421c) having an internal thread and detachably and threadedly connected to the adapter ring 43, and a transparent cover 422 fixed to one end of the positioning end ring (421c) for covering the distal end of the insertion tube 903 of the endoscope 900. The second type of the end ring modules (42c) is similar to the first type, and differs in that the transparent cover 422 of the second type is provided with a plurality of spaced-apart through holes 423 which can respectively correspond to a surgical instrument outlet, a lighting source and a water spout on the distal end of the insertion tube 903. In other implementation of this embodiment, the transparent cover 422 may be omitted.

Through the provision of the adapter ring 43 on the second end portion 412 of the outer sheath 41, and through the provision of the two different types of the end ring modules (42c), after the outer sheath 41 is sleeved on the insertion tube 903, it is convenient for a medical personnel to choose and mount the end ring module (42c) having the transparent cover 422 with or without the through holes 423 according to the current required function of the endoscope 900.

In summary, in the disposable outer sheath mechanism (200, 200′, 200″, 200a, 200b, 200c) of this disclosure, through the structural design of the outer sheath unit (4, 4′, 4c), the fixing unit (3, 3″, 3a, 3b) and the connecting unit (5, 5′), the outer sheath unit (4, 4′, 4c) can be sleeved on the insertion tube 903 of the endoscope 900, so that during use of the endoscope 900, the insertion tube 903 thereof will not be in contact with the body fluids of the patient, or only the distal end of the insertion tube 903 is in contact with the body fluids. Further, the outer sheath unit (4, 4′, 4c) can be directly removed and discarded after use of the endoscope 900. Since the endoscope 900 is not in contact with the body fluids of the patient, it can be reused after being processed by conventional sterilization and disinfection procedures, so that the use and maintenance costs of the endoscope 900 in the medical institution can be greatly reduced.

Moreover, through the detachable structural design of the adapter ring 43 and the two different types of the end ring modules (42c) of the outer sheath unit (4c), it is convenient for a medical personnel to choose and mount the end ring module (42c) having the transparent cover 422 with or without the through holes 423 according to the desired required function of the endoscope 900. Hence, the disposable outer sheath mechanism (200, 200′, 200″, 200a, 200b, 200c) of this disclosure is relatively novel, and is convenient and practical to use. Therefore, the object of this disclosure can indeed be achieved.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A disposable outer sheath mechanism for an endoscope which includes a connecting head and an insertion tube extending outwardly from the connecting head for insertion into a body of a patient, said disposable outer sheath mechanism comprising:
a fixing unit including a socket tube configured to be sleeved on and detachably connected to the connecting head, and a fixing module sleeved on said socket tube for fixing said socket tube to the connecting head;
an outer sheath unit including an outer sheath having an elongated tubular shape and configured to be detachably sleeved on the insertion tube; and
a connecting unit including a first connecting member disposed on one of said socket tube and said outer sheath, and a second connecting member disposed on the other one of said socket tube and said outer sheath for detachable connection with said first connecting member.

2. The disposable outer sheath mechanism as claimed in claim 1, wherein said fixing module includes a C-shaped socket ring sleeved on said socket tube, and a fixing member detachably interconnecting two end portions of said C-shaped socket ring for tightly fixing said socket tube to the connecting head.

3. The disposable outer sheath mechanism as claimed in claim 1, wherein said first connecting member is integrally provided at one end of said socket tube, and said second connecting member has a connecting ring portion sleeved fixedly on said outer sheath, and a plurality of angularly spaced-apart hook portions extending upwardly from said connecting ring portion.

4. The disposable outer sheath mechanism as claimed in claim 3, wherein said first connecting member has an outer peripheral surface provided with an annular engaging groove, and said hook portions are detachably engaged with said annular engaging groove.

5. The disposable outer sheath mechanism as claimed in claim 3, wherein said first connecting member has an outer peripheral surface provided with a plurality of angularly spaced-apart engaging grooves, and said hook portions are respectively and detachably engaged with said engaging grooves.

6. The disposable outer sheath mechanism as claimed in claim 1, wherein said outer sheath has a first end portion configured to be adjacent to the connecting head, and a second end portion configured to be adjacent to a distal end of the insertion tube that is distal to the connecting head.

7. The disposable outer sheath mechanism as claimed in claim 6, wherein said second connecting member is sleeved fixedly on said first end portion of said outer sheath, and said first connecting member is sleeved axially and movably on said socket tube, and is detachably and threadedly connected to said second connecting member.

8. The disposable outer sheath mechanism as claimed in claim 6, wherein said outer sheath unit further includes an end ring module sleeved fixedly on said second end portion of said outer sheath and configured to be sleeved on the distal end of the insertion tube.

9. The disposable outer sheath mechanism as claimed in claim 8, wherein said end ring module includes a positioning end ring sleeved fixedly on said second end portion of said outer sheath.

10. The disposable outer sheath mechanism as claimed in claim 9, wherein said end ring module further includes a transparent cover fixed to one end of said positioning end ring for covering the distal end of the insertion tube.

11. The disposable outer sheath mechanism as claimed in claim 6, wherein said outer sheath unit further includes an adapter ring sleeved fixedly on said second end portion of said outer sheath, and an end ring module detachably connected to said adapter ring and configured to be sleeved on the distal end of the insertion tube.

12. The disposable outer sheath mechanism as claimed in claim 11, wherein said end ring module includes a positioning end ring detachably and threadedly connected to said adapter ring.

13. The disposable outer sheath mechanism as claimed in claim 12, wherein said end ring module further includes a transparent cover fixed to one end of said positioning end ring for covering the distal end of the insertion tube.

14. A disposable outer sheath mechanism for an endoscope which includes a connecting head and an insertion tube extending outwardly from the connecting head for insertion into a body of a patient, said disposable outer sheath mechanism comprising:
an outer sheath unit including an outer sheath having an elongated tubular shape and configured to be detachably sleeved on the insertion tube; and
a fixing unit fixed to said outer sheath and configured to be detachably connected to the connecting head.

15. The disposable outer sheath mechanism as claimed in claim 14, wherein said fixing unit includes a socket tube fixed to said outer sheath and configured to be detachably sleeved on the connecting head, and a fixing module mounted on said socket tube for fixing and positioning said socket tube on the connecting head.

16. The disposable outer sheath mechanism as claimed in claim 15, wherein said fixing module includes a C-shaped socket ring sleeved on said socket tube, and a fixing member detachably two end portions of said C-shaped socket ring for tightly fixing said socket tube to the connecting head.

17. The disposable outer sheath mechanism as claimed in claim 15, wherein said fixing module includes a fixing strap wrapped around said socket tube, and two fixing members respectively disposed on two opposite sides of said fixing strap and detachably connected to each other for tightly fixing said socket tube to the connecting head.

18. The disposable outer sheath mechanism as claimed in claim 14, wherein said outer sheath has a first end portion configured to be adjacent to the connecting head, and a second end portion configured to be adjacent to a distal end of the insertion tube that is distal to the connecting head.

19. The disposable outer sheath mechanism as claimed in claim 18, wherein the connecting head of the endoscope has an outer peripheral surface provided with threads, and said fixing unit includes a fixing ring sleeved fixedly on said first end portion of said outer sheath, and a threaded ring sleeved on and axially movable relative to said fixing ring for threadedly connecting with the threads on the outer peripheral surface of the connecting head.

20. The disposable outer sheath mechanism as claimed in claim 18, wherein the connecting head of the endoscope has an outer peripheral surface provided with threads, and said fixing unit includes a socket tube fixed to said first end portion of said outer sheath and configured to be threadedly and detachably connected to the connecting head.

21. The disposable outer sheath mechanism as claimed in claim 18, wherein said outer sheath further includes an end ring module sleeved fixedly on said second end portion of said outer sheath and configured to be sleeved on the distal end of the insertion tube.

22. The disposable outer sheath mechanism as claimed in claim 21, wherein said end ring module includes a positioning end ring sleeved fixedly on said second end portion of said outer sheath.

23. The disposable outer sheath mechanism as claimed in claim 22, wherein said end ring module further includes a transparent cover fixed to one end of said positioning end ring for covering the distal end of the insertion tube.

24. The disposable outer sheath mechanism as claimed in claim 18, wherein said outer sheath unit further includes an adapter ring sleeved fixedly on said second end portion of said outer sheath, and an end ring module detachably connected to said adapter ring and configured to be sleeved on the distal end of the insertion tube.

25. The disposable outer sheath mechanism as claimed in claim 24, wherein said end ring module includes a positioning end ring detachably and threadedly connected to said adapter ring.

26. The disposable outer sheath mechanism as claimed in claim 25, wherein said end ring module further includes a transparent cover fixed to one end of said positioning end ring for covering the distal end of the insertion tube.
